SEQUENCE LISTING (20 sequences)

SEQ ID NO: 1 - DNA, 25 bp, artificial
  IRS-1 antisens
tctccggagggctcgccatgctgct

SEQ ID NO: 2 - DNA, 25 bp, artificial
  IRS-1 antisens
tatccggagggctcgccatgctgct

SEQ ID NO: 3 - DNA, 24 bp, artificial
  IRS-1 antisens
tctccggagggctcgccatgctgc

SEQ ID NO: 4 - DNA, 23 bp, artificial
  IRS-1 antisens
tctccggagggctcgccatgctg

SEQ ID NO: 5 - DNA, 22 bp, artificial
  IRS-1 antisens
tctccggagggctcgccatgct

SEQ ID NO: 6 - DNA, 21 bp, artificial
  IRS-1 antisens
tctccggagggctcgccatgc

SEQ ID NO: 7 - DNA, 20 bp, artificial
  IRS-1 antisens
tctccggagggctcgccatg

SEQ ID NO: 8 - DNA, 19 bp, artificial
  IRS-1 antisens
tctccggagggctcgccat

SEQ ID NO: 9 - DNA, 24 bp, artificial
  IRS-1 antisens
ctccggagggctcgccatgctgct

SEQ ID NO: 10 - DNA, 23 bp, artificial
  IRS-1 antisens
tccggagggctcgccatgctgct

SEQ ID NO: 11 - DNA, 22 bp, artificial
  IRS-1 antisens
ccggagggctcgccatgctgct

SEQ ID NO: 12 - DNA, 21 bp, artificial
  IRS-1 antisens
cggagggctcgccatgctgct

SEQ ID NO: 13 - DNA, 20 bp, artificial
  IRS-1 antisens
ggagggctcgccatgctgct

SEQ ID NO: 14 - DNA, 19 bp, artificial
  IRS-1 antisens
gagggctcgccatgctgct

SEQ ID NO: 15 - DNA, 18 bp, artificial
  IRS-1 antisens
agggctcgccatgctgct

SEQ ID NO: 16 - DNA, 16 bp, artificial
  IRS-1 antisens
ggctcgccatgctgct

SEQ ID NO: 17 - DNA, 15 bp, artificial
  IRS-1 antisens
gctcgccatgctgct

SEQ ID NO: 18 - DNA, 14 bp, artificial
  IRS-1 antisens
ctcgccatgctgct

SEQ ID NO: 19 - DNA, 13 bp, artificial
  IRS-1 antisens
tcgccatgctgct

SEQ ID NO: 20 - DNA, 12 bp, artificial
  IRS-1 antisens
cgccatgctgct

(19) United States
(12) Patent Application Publication
(10) Pub. No.: US 2013/0143947 A1
(43) Pub. Date: Jun. 6, 2013

(54) Injectable Pharmaceutical Composition for Preventing, Stabilising and/or Inhibiting Pathological Neovascularization-Related Conditions

(71) Applicants: Salman Al-Mahmood, Paris (FR); Sylvie Colin, Paris (FR); Jean-Pascal Conduzorgues, Montpellier (FR); Eric Viaud, Lausanne (CH)

(72) Inventors: Salman Al-Mahmood, Paris (FR); Sylvie Colin, Paris (FR); Jean-Pascal Conduzorgues, Montpellier (FR); Eric Viaud, Lausanne (CH)

(73) Assignee: GENE SIGNAL INTERNATIONALSA, Lausanne (CH)

(21) Appl. No.: 13/814,847

(22) PCT Filed: Aug. 10, 2011
(86) PCT No.: PCT/EP2011/063738

§ 371 Date: Feb. 7, 2013

Related U.S. Application Data
(63) Continuation of application No. 12/853,610, filed on Aug. 10, 2010.

(30) Foreign Application Priority Data
Aug. 10, 2010 (EP) 10305878.0

(51) Int. Cl. A61K 31/711

(52) U.S. Cl. CPC A61K 31/711
USPC 514/44 A; 536/24.5

(57) ABSTRACT

This invention relates to a pharmaceutical composition for the treatment and/or prevention of at least one pathological neovascularization-related conditions of the interior of the eye, the composition comprising a therapeutically effective amount of an antisens oligonucleotide having the sequence SEQ ID NO: 1: 5′-TCTCCGGAGGGCTCGCCATGCTGCT-3′ or any function conservative sequence comprising from 9 to 30 nucleotides that has 75%, 80%, 85%, 90%, 95% or more than 95%, 96%, 97%, 98%, 99% of identity compared to SEQ ID NO: 1 and that conserves the capacity of inhibiting IRS-1 gene expression as SEQ ID NO: 1, and the composition being administered within the posterior segment of the eye to a subject in need thereof; this invention also relates to a method for treating a pathological neovascularization-related condition of the interior of the eye in a subject in need thereof comprising administering to the subject a therapeutically effective amount of said pharmaceutical composition.

### FIELD OF THE INVENTION

This invention relates to the treatment of pathological neovascularization-related conditions, especially in the field of ophthalmology. In particular, this invention relates to an injectable pharmaceutical composition containing an antisense oligonucleotide GS-101, capable of inhibiting the expression of the insulin receptor substrate-1 (IRS-1), to prevent and/or treat ophthalmic pathological neovascularization-related conditions.

### BACKGROUND OF THE INVENTION

Angiogenesis is a fundamental process by means of which new blood vessels are formed. This process is essential in multiple normal physiological phenomena such as reproduction, development and even wound healing.

The formation of neovessels by endothelial cells involves the migration, growth and differentiation of endothelial cells. Regulation of these biological phenomena is linked to Insulin receptor substrate 1 (IRS-1), which is a cytoplasmic docking protein that functions as an essential signalling intermediate downstream of activated cell surface receptors, including insulin, insulin-like growth factor 1 (IGF-1), prolactin, growth hormone (GH), vascular endothelial growth factor (VEGF) receptors, members of the integrin receptor family, and select cytokine receptors.

Even though neovascularisation may be a normal physiological process, pathological neovascularisation is a critical situation in a number of diseases, especially in ophthalmology.

In ophthalmology, pathologies of the posterior segment of the eye, and in particular retinal pathologies, are some of the more disabling pathologies of modern society. Numbered among these pathologies are those characterized by abnormal neovascularization of the retina, iris and choroid, with consequent formation of dysfunctional neovessels which can cause leakage or haemorrhages, or can be associated with retinal edema, retinal/vitreous haemorrhage or retinal detachment resulting in the decline of visual acuity (Survey of Ophthalmology, January 2007, Vol. 52, 51, S3-S19). Ocular pathological neovascularization is also known as one of the leading causes of blindness in humans and is found in diverse eye diseases (Bradley, et al., 2007, Angiogenesis 10:141-8; Chen and Smith, 2007, Angiogenesis 10:133-40; Friedlander et al., 2007, Angiogenesis 10:89-101).

Among the therapies currently being practiced to treat ocular posterior segment disorders, such as uveitis, macular degeneration, macular edema and the like, is intravitreal injection of corticosteroids.

However, corticosteroids have well-known drawbacks, and there is still a need for new therapies for treating the disorders, conditions or diseases of the interior of the eye, which are conditions related to pathological neovascularization.

This invention brings a solution: it was now found that a specific oligonucleotide, called GS-101, already known in topical administration for its capacity of inhibiting IRS-1 gene expression (see for example EP 1 409 672, Bock et al., Ophthalmologe, 2007, 104:336-44; Al-Mahmood et al., The journal of pharmacology and experimental therapeutics, 2009, 329:496-504; Cursiefen et al., Ophthalmology, 2009, 116(9):1630-7) was of particular interest, when administered intraocularly, for the treatment of pathologies of the interior of the eye related to pathological neovascularization. This was surprising as GS-101 effect on endothelial cells in vitro is maximal after 24 h of incubation and is transitory. According to these results, a long term effect of GS-101 was not expected when administered within the posterior segment of the eye.

The intraocular route brings the further advantage to make it possible to administrate a therapeutically effective, but low amount of active ingredient, to the patient. It also has a further advantage to control the exact administered amount, and to avoid any non-compliance to the treatment due to patient.

### SUMMARY OF THE INVENTION

The present invention thus relates to a pharmaceutical composition for use in the treatment and/or prevention of at least one pathological neovascularization-related conditions of the interior of the eye, said composition comprising a therapeutically effective amount of an antisens oligonucleotide having the sequence SEQ ID NO: 1,

5′-TCTCCGGAGGGCTCGCCATGCTGCT-3′

or any function conservative sequence comprising from 9 to 30 nucleotides that has 75%, 80%, 85%, 90%, 95% or more than 95%, 96%, 97%, 98%, 99% of identity compared to SEQ ID NO: 1 and that conserves the capacity of inhibiting IRS-1 gene expression as SEQ ID NO: 1,
and said composition being administered to a subject in need thereof by an administration within the posterior segment of the eye.

In one embodiment of the invention, the function conservative sequence of SEQ ID NO: 1 is 5′-TATCCGGAGGGCTCGCCATGCTGCT-3′ (SEQ ID NO: 2).

In one embodiment of the invention, the function conservative sequence of SEQ ID NO: 1 is selected from the group consisting of:

(SEQ ID NO: 3)
5′-TCTCCGGAGGGCTCGCCATGCTGC-3′
(SEQ ID NO: 4)
5′-TCTCCGGAGGGCTCGCCATGCTG-3′
(SEQ ID NO: 5)
5′-TCTCCGGAGGGCTCGCCATGCT-3′
(SEQ ID NO: 6)
5′-TCTCCGGAGGGCTCGCCATGC-3′
(SEQ ID NO: 7)
5′-TCTCCGGAGGGCTCGCCATG-3′
(SEQ ID NO: 8)
5′-TCTCCGGAGGGCTCGCCAT-3′
(SEQ ID NO: 9)
5′-CTCCGGAGGGCTCGCCATGCTGCT-3′
(SEQ ID NO: 10)
5′-TCCGGAGGGCTCGCCATGCTGCT-3′
(SEQ ID NO: 11)
5′-CCGGAGGGCTCGCCATGCTGCT-3′
(SEQ ID NO: 12)
5′-CGGAGGGCTCGCCATGCTGCT-3′
(SEQ ID NO: 13)
5′-GGAGGGCTCGCCATGCTGCT-3′
(SEQ ID NO: 14)
5′-GAGGGCTCGCCATGCTGCT-3′
(SEQ ID NO: 15)
5′-AGGGCTCGCCATGCTGCT-3′
(SEQ ID NO: 16)
5′-GGCTCGCCATGCTGCT-3′
(SEQ ID NO: 17)
5′-GCTCGCCATGCTGCT-3′
(SEQ ID NO: 18)
5′-CTCGCCATGCTGCT-3′
(SEQ ID NO: 19)
5′-TCGCCATGCTGCT-3′
(SEQ ID NO: 20)
5′-CGCCATGCTGCT-3′

In one embodiment of the invention, the oligonucleotide comprised in the pharmaceutical composition of the invention is in a concentration of about 0.01 mg/ml to about 100 mg/ml.

In one embodiment of the invention, the volume of the injected composition per administration ranges from 1 to 500 μl.

In one embodiment of the invention, the administration within the posterior segment of the eye is an intravitreal injection.

In one embodiment of the invention, the composition as hereinabove described is injected at most once a week, preferably once every two weeks, more preferably once every three weeks, even more preferably once a month, and still even more preferably once every two months.

In one embodiment of the invention, the composition is packaged in the form of unit dose; preferably the unit dose is a disposable syringe. Advantageously, the composition is sterile. According to an embodiment of the invention, the pathological neovascularization-related condition may be uveitis, choroiditis, retinochoroiditis, chorioretinitis, retinal degeneration, AMD, retinal detachment, retinal neovascularisation, proliferative vitreoretinopathy, retinopathy of prematurity (ROP), diabetic retinopathy, posterior segment trauma, retinal vascular pathologies, endophthalmitis, macular edema, inflammatory pathologies of the retina, systemic pathologies with implications for the retina.

### DETAILED DESCRIPTION OF THE INVENTION

The invention thus relates to a pharmaceutical composition for treating or for use in treating ocular disorders of the interior of the eye, linked to pathological neovascularization, wherein said composition comprises a therapeutically effective amount of GS-101 and said composition is administered to a subject in need thereof, by intraocular route. In one embodiment of the invention, said intraocular route refers to an administration within the interior of the eye, preferably within the posterior segment of the eye, more preferably an administration within the vitreous. Preferably, said administration is an injection.

In one embodiment, the ocular disorder treated by the composition of the invention is a disorder of the retina, linked to pathological retinal vascularization.

According to the invention, the composition is formulated in a form suitable for intraocular injections. According to a first embodiment, the composition is an aqueous solution. Examples of aqueous solutions include, but are not limited to, a solution of GS-101 in NaCl, preferably in 0.9% NaCl. According to a second embodiment the composition is within an implant. According to a third embodiment, the composition is associated with a sustained delivery system, composition or device. According to a fourth embodiment, the composition may include GS101 and a polymeric agent.

According to the invention, GS-101 is an antisens oligonucleotide having the sequence SEQ ID NO: 1,

5′-TCTCCGGAGGGCTCGCCATGCTGCT-3′

or any function conservative sequence comprising from 9 to 30 nucleotides that has 75%, 80%, 85%, 90%, 95% or more than 95%, 96%, 97%, 98%, 99% of identity compared to SEQ ID NO: 1 and that conserves the capacity of inhibiting pathological neovascularization as SEQ ID NO: 1.

The term “identity” or “identical”, when used in a relationship between the sequences of two or more nucleotidic sequences, refers to the degree of sequence relatedness between nucleotidic sequences, as determined by the number of matches between strings of two or more bases. “Identity” measures the percent of identical matches between the smaller of two or more sequences with gap alignments (if any) addressed by a particular mathematical model or computer program (i.e., “algorithms”). Identity of related nucleotidic sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An example of a function conservative sequence of SEQ ID NO: 1 is SEQ ID NO: 2 (5′-TATCCGGAGGGCTCGCCATGCTGCT-3′). Other examples of a function conservative sequence of SEQ ID NO: 1 are the following sequences:

(SEQ ID NO: 3)
5′-TCTCCGGAGGGCTCGCCATGCTGC-3′
(SEQ ID NO: 4)
5′-TCTCCGGAGGGCTCGCCATGCTG-3′
(SEQ ID NO: 5)
5′-TCTCCGGAGGGCTCGCCATGCT-3′
(SEQ ID NO: 6)
5′-TCTCCGGAGGGCTCGCCATGC-3′
(SEQ ID NO: 7)
5′-TCTCCGGAGGGCTCGCCATG-3′
(SEQ ID NO: 8)
5′-TCTCCGGAGGGCTCGCCAT-3′
(SEQ ID NO: 9)
5′-CTCCGGAGGGCTCGCCATGCTGCT-3′
(SEQ ID NO: 10)
5′-TCCGGAGGGCTCGCCATGCTGCT-3′
(SEQ ID NO: 11)
5′-CCGGAGGGCTCGCCATGCTGCT-3′
(SEQ ID NO: 12)
5′-CGGAGGGCTCGCCATGCTGCT-3′
(SEQ ID NO: 13)
5′-GGAGGGCTCGCCATGCTGCT-3′
(SEQ ID NO: 14)
5′-GAGGGCTCGCCATGCTGCT-3′
(SEQ ID NO: 15)
5′-AGGGCTCGCCATGCTGCT-3′
(SEQ ID NO: 16)
5′-GGCTCGCCATGCTGCT-3′
(SEQ ID NO: 17)
5′-GCTCGCCATGCTGCT-3′
(SEQ ID NO: 18)
5′-CTCGCCATGCTGCT-3′
(SEQ ID NO: 19)
5′-TCGCCATGCTGCT-3′
(SEQ ID NO: 20)
5′-CGCCATGCTGCT-3′

According to an embodiment, said function conservative sequence comprising 9 to 30 nucleotides may be a sequence comprising SEQ ID NO: 1 or SEQ ID NO: 2 between other nucleic acids in C-terminal and N-terminal. Said function conservative sequence may also be a 9 to 12 contiguous nucleotides fragment of SEQ ID NO: 1 or SEQ ID NO: 2.

The pharmaceutical composition may include GS-101 as described above, or a function conservative sequence thereof, as described above, in association with any pharmaceutically acceptable excipients for intraocular route. In one embodiment of the invention, said intraocular route is an administration within the interior of the eye, preferably within the posterior segment of the eye, more preferably an administration within the vitreous. Preferably, said administration is an intravitreal injection.

The present invention also relates to a method for treating a pathological neovascularization-related condition of the interior of the eye in a subject in need thereof comprising administering to the subject a therapeutically effective amount of a composition comprising GS-101 as described above, or a function conservative sequence thereof, as described above, in association with a pharmaceutically acceptable excipient, and said composition is administered by intraocular route. In one embodiment of the invention, said intraocular route is an administration within the interior of the eye, preferably within the posterior segment of the eye, more preferably an administration within the vitreous. Preferably, said administration is an intravitreal injection.

In one embodiment, said pathological neovascularization-related condition of the interior of the eye is selected among uveitis, choroiditis, retinochoroiditis, chorioretinitis, retinal degeneration, AMD, retinal detachment, retinal neovascularization, proliferative vitreoretinopathy, retinopathy of prematurity (ROP), diabetic retinopathy, posterior segment trauma, retinal vascular pathologies, endophthalmitis, macular edema, inflammatory pathologies of the retina, and systemic pathologies with implications for the retina.

According to an embodiment, GS-101 as described above, or a function conservative sequence thereof, is present in the composition of the invention in a concentration of about 0.01 mg/ml to about 100 mg/ml, preferably 0.1 mg/ml to 80 mg/ml, more preferably 6 to 60 mg/ml, and even more preferably from about 8 to about 12 mg/ml.

According to an embodiment, the volume of the injected composition per administration ranges from 1 to 500 μl, preferably from about 5 to 400 μl, more preferably from 5 to 300 μl. According to an embodiment, the volume of the injected composition is of about 50 μl.

According to an embodiment, the amount of GS-101 injected per administration ranges from 10 to 5000 μg, preferably from about 50 to 4000 μg, more preferably from 50 to 3000 μg. According to an embodiment, the amount of GS-101 injected is of about 500 μg.

In one embodiment, the pharmaceutical composition is such that the intraocular administration is intravitreal administration.

According to an embodiment, the composition is injected at most once every three weeks, preferably at most once every four weeks, even more preferably once every eight weeks.

As shown in the Examples, the composition of the invention results in an optimal treatment with at most one injection every three weeks. This result is surprising, as, when endothelial cells are incubated in vitro with GS-101, the effect is maximal 24 hours after incubation, and is only transitory.

According to an embodiment, the patient is treated with one injection every three weeks, preferably one injection every four weeks during two to five, preferably three months.

According to an embodiment, the duration of the treatment is of three to six months. The treatment may be renewed if a loss of visual acuity is observed in the patient.

In one embodiment of the invention, said pharmaceutical composition as described here above is packaged in the form of unit dose.

In another embodiment, said unit dose is a disposable syringe.

Advantageously, said unit dose is sterile.

### DEFINITIONS

According to this invention, the following terms have the following meanings:

“About” means plus or minus ten percent of the number, parameter or characteristic so qualified.

“Interior of the eye” means any area located within the eyeball, including the anterior and posterior segment of the eye, and which generally includes, but is not limited to, any functional (e.g., for vision) or structural tissues found within the eyeball, or tissues or cellular layers that partly or completely line the interior of the eyeball. Specific examples of areas include the anterior chamber, the posterior chamber, the vitreous cavity, the choroid, the macula, and the retina, and blood vessels and nerves which vascularize or innervate a posterior ocular region or site. According to a preferred embodiment, interior of the eye means the posterior segment of the eye, including the posterior chamber, the vitreous cavity, the choroid, the macula, and the retina, and blood vessels and nerves which vascularize or innervate a posterior ocular region or site. Preferably, the expression “a disease of the interior of the eye”, refers to a disease of the retina and/or choroid and/or macula and/or the posterior chamber.

“Conditions related to pathological neovascularization” or “pathological neovascularization-related conditions” are diseases where undesired neovascularization is present, and include the following: uveitis, choroiditis, retinochoroiditis, chorioretinitis, retinal degeneration, AMD, retinal detachment, retinal neovascularization, proliferative vitreoretinopathy, retinopathy of prematurity (ROP), diabetic retinopathy, posterior segment trauma, retinal vascular pathologies, endophthalmitis, macular edema, inflammatory pathologies of the retina, systemic pathologies with implications for the retina, possibly in combination with other therapies for the treatment of the same pathologies. Especially, the invention aims at addressing blood and lymph neovascularization.

“Treating a disease” means preventing (i.e. keeping from happening), reducing or alleviating at least one adverse effect or symptom of a disease, disorder or condition associated with a deficiency in or absence of an organ, tissue or cell function.

“Therapeutically effective amount” means level or amount of agent that is aimed at, without causing significant negative or adverse side effects to the target, (1) delaying or preventing the onset of a disease, disorder, or condition related to pathological neovascularization; (2) slowing down or stopping the progression, aggravation, or deterioration of one or more symptoms of the disease, disorder, or condition related to pathological neovascularization; (3) bringing about ameliorations of the symptoms of the disease, disorder, or condition related to pathological neovascularization; (4) reducing the severity or incidence of the disease, disorder, or condition related to pathological neovascularization; or (5) curing the disease, disorder, or condition related to pathological neovascularization. A therapeutically effective amount may be administered prior to the onset of the disease, disorder, or condition related to pathological neovascularization, for a prophylactic or preventive action. Alternatively or additionally, the therapeutically effective amount may be administered after initiation of the disease, disorder, or condition related to pathological neovascularization, for a therapeutic action. In one embodiment, the target is the interior of the eye; in this embodiment, a therapeutically effective amount of GS-101 is an amount that is effective in reducing at least one symptom of a pathological neovascularization-related condition of the interior of the eye.

“Intraocularly” means by intraocular route of administration. In one embodiment, the intraocular route of administration is an administration within the interior of the eye, preferably within the posterior segment of the eye, more preferably within the vitreous. Preferred intraocular route is intravitreal injection. According to the present invention, the intraocular route is of specific interest, in that it results in administering a rather low, and therapeutically accurate dose of active ingredient (i.e. GS-101 or function conservative sequence thereof) and result in an optimal treatment. Indeed, as shown in the Examples, the composition of the invention results in an optimal treatment with at most one injection every three weeks. This result is surprising, as, when cells are incubated with GS-101, the effect is maximal 24 hours after incubation, and is only transitory.

“Subject” refers to a mammal, preferably a human, but also can refer to animals, such as for example pets.

### DESCRIPTION OF THE FIGURES

FIG. 1 is a histogram showing the effects of two intraocular injections of GS-101 or its vehicle (NaCl) on neovascularization of the retina following OR. Results are mean±SEM. *: P<0.01 compared to vehicle; 1:: P<0.05 compared to Scramble. (Scramble is an oligonucleotide with same bases as GS-101, but not with the same sequence, from which was designed the term “scramble”).

FIG. 2 is a histogram showing the effect of an intravitreal injection of GS-101 on lesion late phase fluorescein signal intensity at day 22/23 post-laser treatment. Vertical bars indicate SEM.

FIG. 3 is a histogram showing the effects of treatment of human endothelial cells with a single dose of GS-101 (10 μM) or scramble oligonucleotide (10 μM) onto IRS-1 expression over-time. IRS-1 proteins were quantified by ELISA. Results were presented as % of control (cells incubated with vehicle) and its represent means in three experiments. Vertical bars indicate SEM. ns=not shown.

### EXAMPLES

### Example 1

### Experimental Approaches

The procedures and protocols were approved by our institutional review board and were performed in accordance with our institutional guidelines and the Guide for the Care and Use of Laboratory Animals of France and Canada. Animals were kept under standard conditions with free access to food and water (24° C.; 12:12 hr light/dark cycle).

### Oxygen-Induced Retinopathy in Rats

Oxygen-induced retinopathy (OIR) rat model was used as previously described (Dorfman A, Dembinska O, Chemtob S, Lachapelle P. Early manifestations of postnatal hyperoxia on the retinal structure and function of the neonatal rat. Invest Ophthalmol Vis Sci 2008; 49:458-466). Newborn litters of Sprague-Dawley rats (Charles River Laboratories, St-Constant, Quebec, Canada) were exposed to 80% oxygen immediately after birth (mixture of medical grade 100% O₂ and room air measured with an oxygen meter; MaxO₂ Ceramatec, model OM25-ME; Medicana Inc., Montreal, Quebec, Canada). Briefly, exposure to hyperoxia (80% O₂) persisted from birth until postnatal day 14 for 22.5 hours daily, interrupted with three intervals of 30 minutes' duration under normoxic conditions (21% O₂). After hyperoxic exposure, animals were assigned at P14 to the following experiment, animals were treated at P14 and P16 by an intravitreal injection of 1 μl in one eye of either the vehicle alone (sterile NaCl 0.9%) (n=8), or containing either a scramble GS-101 oligonucleotide (2 mg/ml, 2 μg delivered, n=4), or GS-101 (SEQ ID NO: 2) at the concentration of 0.5 mg/ml (0.5 μg delivered, n=7), 1 (1 μg delivered, n=8), and 2 (2 μg delivered, n=8). Before the injection, rats were anesthetized with halothane (˜2.5%) and injected with a 10 μl-Hamilton syringe attached to a glass capillary of approximately 60 gauge. During the treatment period, rats were maintained in a cyclic lighting environment (80 lux; 12 hours dark/12 hours light). Finally, mothers of the litters were alternated between normoxic and hyperoxic conditions every 24 hours so that pulmonary complications known to arise in adult rats raised in a hyperoxic environment could be avoided. All animals were then euthanized at P18: the eyes were enucleated, the anterior segments dissected, and the eyecups fixed overnight in 4% formalin. Doses detailed above are based on the estimated eye volumes (˜25 μl in third postnatal week vs. ˜50 μl in adult).

### Developmental Retinal Vascularization in Newborn Rats

Under halothane anesthesia, 1 μl in one eye of either the vehicle alone (sterile NaCl 0.9%) (n=6), or containing GS-101 at the concentration of 0.5 mg/ml (0.5 μg delivered, n=8) was injected at P1 and P3. Rat pup eye volume is approximately 0.3% of that of human adult.

### Retinal Flatmounts

The retinas were subsequently isolated, and flatmounts were prepared for staining with adenosine diphosphatase (ADPase). Specimens were mounted and photographed (40×, Axiophot microscope; Carl Zeiss Meditec, GmbH, Oberkochen, Germany). For the developmental protocol, retinal vascularization area and density have been evaluated using ImagePro Plus 4.5 (Media Cybernetics, Silver Spring, Md.). For the OIR protocol, the severity of retinopathy has been assessed using a retinal scoring system which evaluates the following criteria: blood vessel growth, blood vessel tufts, extra-retinal neovascularization, central vasoconstriction, retinal hemorrhage, and blood vessel tortuosity. In addition, vascular tufts per se have been evaluated on retinal flatmounts.

### Statistical Analysis

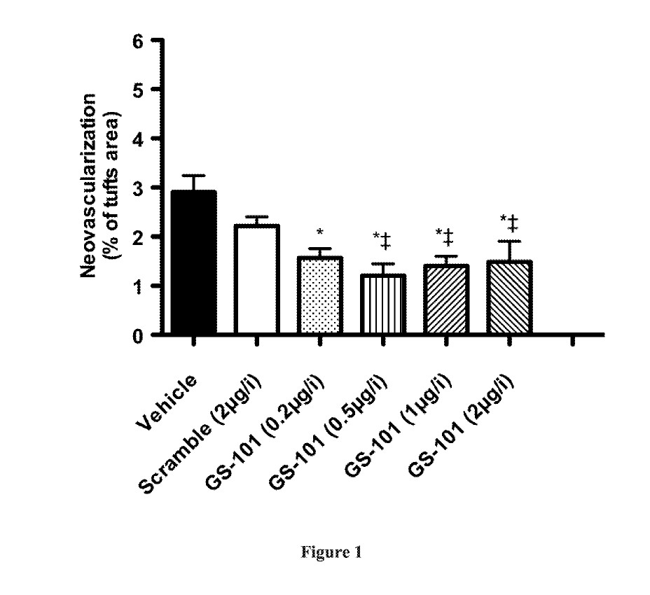

Statistical analyses were made by ANOVA using the Dunnett posttest (GraphPad Software Inc, San Diego, Calif., USA). P<0.05 were considered significant. To determine the effective concentration of GS-101 required in the posterior chamber to inhibit retinal neovascularization in rats with OIR, GS-101 was injected in the eye. As shown by FIG. 1, a dose of 0.5 μg reduced pathological neovascularization. Considering the volume of the eye (25 μl), this suggests that a concentration of 20 μg/ml is required to reduce retinal neovascularization in the rat model of OIR

### Results

TABLE 1
Effects of intra-ocular injection of GS-101 on normal vascularization
and its density in the retina during development in newborn rats.

| | Vehicle | | GS-101 0.5 μg/injection | |
|---|---|---|---|---|
| | Vasc Area (%) | Density (%) | Vasc Area (%) | Density (%) |
| Moy | 53.60 | 39.85 | 51.87 | 34.23 |
| SEM | 1.77 | 1.66 | 2.76 | 2.46 |
| N | 6 | 7 | 8 | 8 |

Vasc Area: vascular area.

As demonstrated in table 1, intraocular injections of GS-101 does not alter the normal evolution of the vascularization of the retina at concentration that have been shown to be effective in the rat model of OIR (FIG. 1). Hence, GS-101 confirms its safety profile.

### Example 2

Experimental Approaches

In this study, laser-induced choroidal neovascularization (CNV) in African green monkeys was used as a model of wet AMD to assess the efficacy of GS-101.

### Test Compound

Test article GS-101 (SEQ ID NO: 2) was provided by Crid Pharma (St. Gely du Fesc, France) suspended in saline (10 μg GS-101/1 μl 0.9% NaCl solution).

### Laser-Induced Choroidal Neovascularization in Monkeys

Three male adult African green monkeys ranging in weight from 4.15 to 5.81 kg, estimated age: 4-12 years old, were used in the study. All animals received on day 1 six laser burns concentrically spaced approximately 1.5 disc diameters from the fovea at the anatomic periphery of the macula, within the temporal vascular arcades of both eyes. Laser spots were applied using an Iridex Oculight TX 532 nm laser with laser energy of 800 mW, pulse duration at 100 ms, and spot size at 50 μm. Fundus photographs were reviewed to confirm perimacular placement of laser lesions.

A sterile solution of GS-101 at 10 μg/μl was administered intravitreally (IVT) with an injection volume of 50 μl, immediately following laser photocoagulation. Eyes were examined by direct external inspection, slit lamp biomicroscopy and indirect retinoscopy at baseline and day 22. Fundus images and angiograms were collected on OS (left eye) followed by OD (right eye) on days 22-23.

### Graded Scoring of Angiograms

Graded scoring of angiograms was performed on fluorescein angiogram series collected on days 22/23, performed on successive days to allow clearance of fluorescein from ocular tissues between angiogram series. The extent of late phase fluorescein leakage at each lesion site, an angiographic change associated with CNV, was rated on a Ito IV scale with:
I=no hyperfluorescence
II=hyperfluorescence without leakage and no significant residual staining in late phase angiograms
III=hyperfluorescence early of mid-transit with late leakage and significant residual staining
IV=hyperfluorescence early or mid-transit with late leakage extending beyond the borders of the treated area.
   Scoring was conducted by two investigators who remained masked to treatment group.

### Imagej Scoring of Angiograms

Original angiogram jpg files were further assessed using Image J (Rasband W. S., ImageJ, U.S. National Institutes of Health, Bethesda, Md., USA). Relative late-phase fluorescein intensity was quantified by assessment of background-corrected spot intensity in angiograms.

### Statistical Methods

Graded (I-IV) scoring of lesions was analyzed using the Fisher's exact probability test where incidence of either grade IV, grade III or both grade III and IV lesions was assigned ‘Yes’ and any other grading assigned ‘No’, comparing to historical vehicle control data. Incidence of either grade I, grade II, or both grade I and II was evaluated in identical fashion.

Validation of the laser-induced CNV model has demonstrated that, similar to other CNV modeling studies in non-human primates, clinically significant CNV is represented by grade III and grade IV lesion scores. The effect of laser power on incidence of CNV was compared at day 22/23 between eyes receiving GS-101 and vehicle. ImageJ based analysis of background-corrected mean fluorescein intensity and CNV complex area were analyzed using one-way ANOVA on Box-Cox-transformed values with post-hoc analysis using Turkey-Kramer HSD. All statistics were performed using the statistical analysis software JMP(SAS Institute, Cary, N.C.). P values<0.5 were considered statistically significant.

### Results

A significantly lower incidence of grade III/IV CNV lesions was observed in eyes receiving IVT GS-101 than those receiving vehicle (p=0.000183), as shown in Table 2 below. Table 2 illustrates overall results of angiogram scoring.

TABLE 2
Lesion scoring summary

% of total number lesions

| Treatment | Grade I | Grade II | Grade III | Grade IV |
|---|---|---|---|---|
| Vehicle | 0 | 46.6 | 32.8 | 20.7 |
| IVT GS-101 | 13.3 | 60 | 26.7 | 0 |
| (10 μg/μl) | | | | |

Evaluating incidence of grade II lesions, representing lower intensity CNV, also demonstrated a lower incidence in eyes receiving IVT GS-101 versus those receiving vehicle (p=0.006545). In eyes receiving IVT GS-101, no grade IV lesions remained at day 22/23 compared with control eyes. Furthermore, incidence of grade III lesions was strongly reduced.

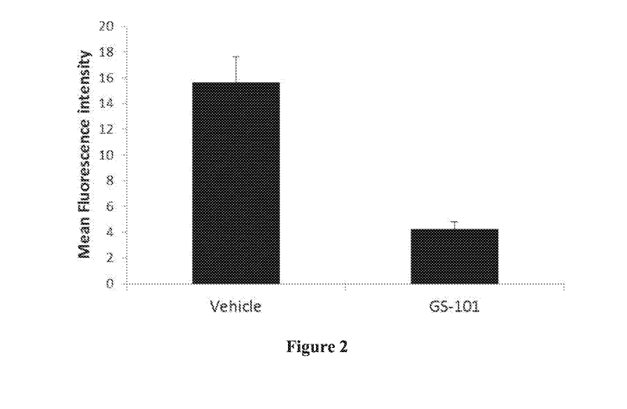

CNV development was assessed by ImageJ analysis of fluorescein angiograms to provide comparison of late phase relative fluorescein intensity in lesions from the IVT GS-101 and vehicle-treated eyes. As shown in FIG. 2, IVT delivery of GS-101 elicited a significant decrease in late phase fluorescein signal intensity compared with eyes receiving the vehicle (p=0.0000025).

These effects of GS-101 reveal a prominent anti-angiogenic effect of direct delivery to the posterior pole of the eye. These findings suggest that IVT treatment with GS-101 may provide a potent anti-angiogenic treatment for wet AMD and other neovascular retinal diseases.

### Example 3

### Experimental Approaches

Cell Culture and Treatments

Human endothelial cells (hEC) were cultured in complete EGM-2MV. At about 80% of confluence, culture medium was discarded, and cell layer was washed three times with serum deprived culture medium. Cell layers were then incubated over night with culture medium containing 1% serum at 37° C. under 5% CO₂.

After incubation, GS-101 (SEQ ID NO: 2 of the invention) or scramble oligonucleotide (in PBS) was added to the culture at 10 μM final concentration, and the incubation was furthered for the indicated time.

### Protein Quantification

Human EC were incubated with GS-101 or scrambled oligonucleotides at 37° C. under 5% CO₂ as above. Following the indicated time of incubation, cells were washed three times with ice-cold PBS, and suspended in the protein extraction buffer (PEB) (20 mM Tris-HCl pH 7.5, 150 mM NaCl, 1 mM EDTA, 1 mM EGTA, 1% Triton, 25 mM sodium pyrophosphate, 1 mM β-glycero-phosphate, 1 mM Na3Vo4, 1 μg/ml leupeptine, 1 μM PMSF). The protein content was measured by Bradford. IRS-1 concentration in the extracts was determined by Path-Scan Total IRS-1 Sandwich ELISA kit (Cell Signaling technology) according to the manufacturer's instructions. The data were collected from 3 separate experiments performed in duplicate and expressed relative to control cells (cells incubated with vehicle).

### Results

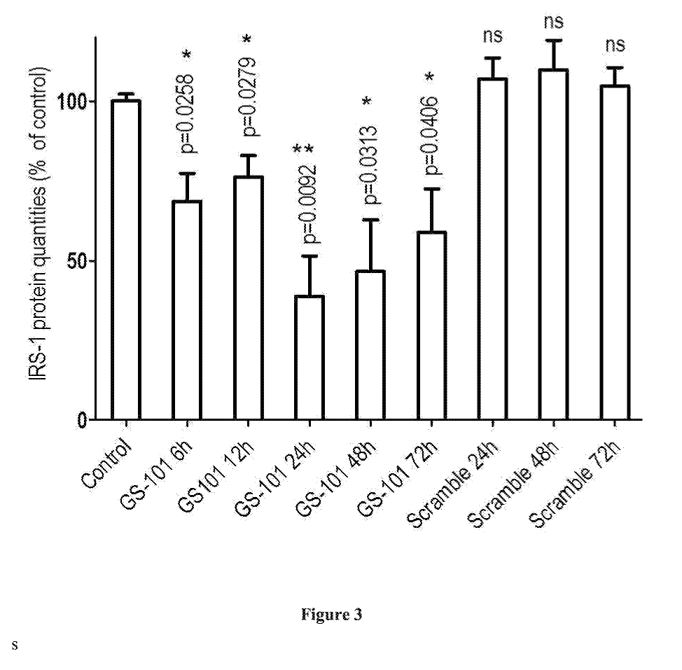

The results of IRS-1 protein quantifications by ELISA are presented in FIG. 3. These results showed that human endothelial cells treated with a single dose of GS-101 (10 μM) contained:
31.40±9.071% (p=0.0258; N=3) less IRS-1 protein contents relative to control following 6 h of incubation;
23.80±7.052% (p=0.0279; N=3) less IRS-1 protein contents relative to control following 12 h of incubation;
61.30±12.99% (p=0.0092; N=3) less IRS-1 protein contents relative to control following 24 h of incubation;
53.50±16.45% (p=0.0313; N=3) less IRS-1 protein contents relative to control following 48 h of incubation;
41.10±13.78% (p=0.0406; N=3) less IRS-1 protein contents relative to control following 72 h of incubation.

No significant variations (p>0.05) in IRS-1 protein contents of human endothelial cells treated with the scramble oligonucleotide at 10 μM was observed.

These results show that inhibition of IRS-1 is maximal 24 h after GS-101 incubation and is transitory. These in vitro results therefore would not have induced to use GS-101 antisens in an intravitreal injection as the effect of the antisens is transitory and thus limited.

1.-11. (canceled)

12. A pharmaceutical composition comprising an effective amount of an antisense oligonucleotide having the sequence SEQ ID NO: 1:
5′-TCTCCGGAGGGCTCGCCATGCTGCT-3′
or any function conservative sequence comprising from 9 to 30 nucleotides that has 75%, 80%, 85%, 90%, 95% or more than 95%, 96%, 97%, 98%, 99% of identity compared to SEQ ID NO: 1 and that conserves the capacity of inhibiting IRS-1 gene expression as SEQ ID NO: 1, wherein the effective amount of the oligonucleotide is therapeutically effective for the treatment and/or prevention of at least one pathological neovascularization-related condition of the interior of the eye when it is administered to a subject in need thereof by an administration within the posterior segment of the eye.

13. The pharmaceutical composition of claim 1, wherein the function conservative sequence of SEQ ID NO: 1 is 5′-TATCCGGAGGGCTCGCCATGCTGCT-3′ (SEQ ID NO: 2).

14. The pharmaceutical composition of claim 1, wherein the function conservative sequence of SEQ ID NO: 1 is:
(SEQ ID NO: 3)
5′-TCTCCGGAGGGCTCGCCATGCTGC-3′
(SEQ ID NO: 4)
5′-TCTCCGGAGGGCTCGCCATGCTG-3′
(SEQ ID NO: 5)
5′-TCTCCGGAGGGCTCGCCATGCT-3′
(SEQ ID NO: 6)
5′-TCTCCGGAGGGCTCGCCATGC-3′
(SEQ ID NO: 7)
5′-TCTCCGGAGGGCTCGCCATG-3′
(SEQ ID NO: 8)
5′-TCTCCGGAGGGCTCGCCAT-3′
(SEQ ID NO: 9)
5′-CTCCGGAGGGCTCGCCATGCTGCT-3′
(SEQ ID NO: 10)
5′-TCCGGAGGGCTCGCCATGCTGCT-3′
(SEQ ID NO: 11)
5′-CCGGAGGGCTCGCCATGCTGCT-3′
(SEQ ID NO: 12)
5′-CGGAGGGCTCGCCATGCTGCT-3′
(SEQ ID NO: 13)
5′-GGAGGGCTCGCCATGCTGCT-3′
(SEQ ID NO: 14)
5′-GAGGGCTCGCCATGCTGCT-3′
(SEQ ID NO: 15)
5′-AGGGCTCGCCATGCTGCT-3′
(SEQ ID NO: 16)
5′-GGCTCGCCATGCTGCT-3′
(SEQ ID NO: 17)
5′-GCTCGCCATGCTGCT-3′
(SEQ ID NO: 18)
5′-CTCGCCATGCTGCT-3′
(SEQ ID NO: 19)
5′-TCGCCATGCTGCT-3′
(SEQ ID NO: 20)
5′-CGCCATGCTGCT-3′.

15. The pharmaceutical composition of claim 1, further defined as comprising the oligonucleotide in a concentration of about 0.01 mg/ml to about 100 mg/ml.

16. The pharmaceutical composition of claim 1, wherein the amount of the oligonucleotide is comprised in the composition such that it is administered to a subject in a volume of injected composition per administration ranging from 1 to 500 μl.

17. The pharmaceutical composition of claim 1, wherein the composition is packaged in the form of unit dose.

18. The pharmaceutical composition of claim 1, wherein the composition is sterile.

19. The pharmaceutical composition of claim 17, wherein the unit dose is a disposable syringe.

20. The pharmaceutical composition of claim 1, wherein the pathological neovascularization-related condition is uveitis, choroiditis, retinochoroiditis, chorioretinitis, retinal degeneration, AMD, retinal detachment, retinal neovascularisation, proliferative vitreoretinopathy, retinopathy of prematurity (ROP), diabetic retinopathy, posterior segment trauma, retinal vascular pathologies, endophthalmitis, macular edema, inflammatory pathologies of the retina, and/or systemic pathologies with implications for the retina.
